(19) United States
(12) Patent Application Publication
(10) Pub. No.: US 2024/0141020 A1
(43) Pub. Date: May 2, 2024

(54) METHOD FOR MODULATING AFUCOSLYATION OF AN ANTIBODY PRODUCT

(71) Applicants: LONZA BEND INC., Bend, OR (US); LONZA BIOLOGICS PLC, Slough (GB)

(72) Inventors: Julia ARMSTRONG, Bend, OR (US); James GRAHAM, Slough (GB)

(21) Appl. No.: 18/264,831

(22) PCT Filed: Mar. 11, 2021
(86) PCT No.: PCT/US2021/021901

§ 371 Date: Aug. 9, 2023

Related U.S. Application Data
(60) Provisional application No. 63/148,746, filed on Feb. 12, 2021.

(51) Int. Cl. C07K 16/00

(52) U.S. Cl. CPC C07K 16/00; C07K 2317/14; C07K 2317/41

(57) ABSTRACT

The present application is directed to methods for modulating afucosylation of an antibody product that is produced in a bioreactor. The methods include the addition of mannose to the bioreactor to control the afucosylation of the antibody product, including increasing by afucosylation by 1% or more, relative to untreated bioreactor products.

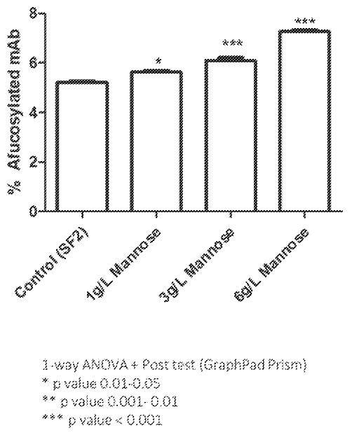

### FIELD OF THE INVENTION

The present application is directed to methods for modulating afucosylation of an antibody product that is produced in a bioreactor. The methods include the addition of mannose to the bioreactor to control the afucosylation of the antibody product, including increasing by afucosylation by 1% or more, relative to untreated bioreactor products.

### BACKGROUND

Protein products, such as antibodies, undergo post-translational modifications during their expression from a cell, including the attachment of sugar moieties. One such modification is N-linked glycosylation of immunoglobulin G (IgG) that occurs at position Asn 297 of the CH2 domain of mammalian IgG heavy chains. This particular N-linked glycosylation is achieved by the initial addition of a pre-formed oligosaccharide which is then subject to subsequent modification to remove glucose and mannose residues and add other sugars such as fucose, galactose, sialic acid and N-acetylglucosamine (GlcNAc). Such glycosylation can have strong effects on the biological activity of the protein. In particular, antibody-dependent cellular cytotoxicity (ADCC), which is an important mechanism of action of many therapeutic antibodies, is dependent on the level of fucosylation of the antibody. Various reports have found that monoclonal antibodies having a reduced amount of fucosylation (i.e., higher afucosylation) exhibit higher ADCC as compared to their fucosylated counterparts. Thus, production of antibody products in which afucosylation can be increased is advantageous for some therapeutic approaches, especially in oncology.

Further, since the type and extent of glycosylation can affect biological activity, the glycan profile of a therapeutic antibody is an important critical quality attribute that must be reported to regulatory authorities and consistently reproduced. However when the manufacture of a therapeutic antibody is transferred from one process to another (or even between manufacturing sites), variations can occur to CQAs such as fucosylation that may result in the need to tune the transferred process to achieve the level of fucosylation in the previously obtained range for that product.

There is a need therefore, to be able to modulate the level of fucosylation of in recombinantly expressed antibody compositions to enhance biological activity and/or to match the levels of fucosylation to a previous manufacturing process.

### SUMMARY

It has been surprisingly found that the addition of mannose, in amounts greater than about 1 g/L, to an antibody production process result in a statistically significant increase in afucosylation of the antibody product (when compared to the same production process without the addition of mannose). Furthermore, the addition of mannose at such amounts in the present methods does not appear to result in an increase in high mannose species. These results are surprising and unexpected, and as described herein, the present methods provide a means to (i) modulate the afucosylation of an antibody product so as to produce a therapeutic antibody that can enhance ADCC, and thus, provide an enhanced therapeutic effect; and (ii) tune afucosylation levels to ensure consistent levels taking into account that this is a critical quality attribute. The present methods have significant advantages over other methods that require genetically engineering cell lines (difficulty in producing and maintaining such cell lines) to enhance afucosylation, as well as other methods that require significant manipulations to upstream bioprocessing parameters.

Accordingly, in one embodiment, the present invention provides a method for increasing afucosylation of an antibody recombinantly expressed in a bioreactor, the method comprising culturing cells that express the antibody in a bioreactor, adding mannose to cell culture during the antibody production process such that the afucosylation of the antibody is increased as compared to the same antibody produced by the antibody production process without the addition of mannose.

Also provided herein are methods for matching the afucosylation of a recombinantly produced antibody to a previously obtained target afucosylation percentage for the same antibody, the method comprising culturing cells that express the antibody in a bioreactor, controlling the addition of mannose to the bioreactor during the antibody production process to obtain the expressed antibody with the target percentage afucosylation.

Other features and aspects of the present disclosure are discussed in greater detail below.

### DETAILED DESCRIPTION

It is to be understood by one of ordinary skill in the art that the present discussion is a description of exemplary embodiments only, and is not intended as limiting the broader aspects of the present disclosure.

In embodiments, provided herein is a method for modulating afucosylation of an antibody product, which is typically intended to (i) modify the biological activity of the antibody e.g. increase antibody-dependent cellular cytotoxicity (ADCC) or (ii) fine-tune the afucosylation in a process to match the levels obtained previously.

“Fucosylation,” a type of glycosylation, is the process of adding fucose sugar units to a molecule, including proteins such as antibodies (also called “antibody products” herein). “Afucosylation,” as used herein, refers to the absence of fucose sugar units on a particular molecule, such as a particular antibody product. In a preparation of antibody product, the level of afucosylation is the percentage of antibody molecules that lack a fucose sugar unit. This can be determined by a number of methods including mass spectrometry and high pressure liquid chromatography (HPLC). Since there can be some variation between the methods used, in one embodiment the percentage is measured using a Time of Flight, Liquid Chromatograph Mass Spectrometer (TOF LC/MS) system. The antibodies are reduced and then loaded directly onto a liquid chromatograph mass spectrometer (LC/MS), such as an Agilent 6230B TOF LC/MS system (without the need for a PNGase F digestion), with the reduced antibodies being passed through a reverse phase de-salting column on the liquid chromatograph prior to being injected into the time of flight mass spectrometer.

Other suitable methods include those described in, for example, Tay and Butler, 2015, J. Biol. Methods 2:19 Mishra et al., 2020, J. Biotechnology X 5: 100015. In one of these described methods, glycans can be removed using PNGase F and dried. They are then labelled using 2-AB labelling and analysed using hydrophilic interaction liquid chromatography-HPLC (HILIC-HPLC).

Detected species are then analysed to determine the percentage of afucosylated antibody. Measurements are usually replicated to improve measurement precision.

In one embodiment, the percentage fucosylation is calculated with respect to only N-linked glycan species, such as N-linked glycan species that are linked to the Fc domain of an antibody. N-linked glycan species include G0, G0F, G1F, G2F, G1F+NeuAc, G2F+NeuAc, G2F+2NeuAc. In one embodiment, afucosylation is measured as a percentage based on G0/(G0+G0F). Measurements based on G0 and G0F can be preferred since other isoforms can be difficult to resolve or co-elute with high mannose forms.

As used herein, an “antibody product” and “antibody” are used interchangeably, with antibody product being the result of an antibody production process. As used herein, the terms “antibody” and “immunoglobulin” can be used interchangeably and refer to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, with two pairs of polypeptide chains, each pair having one “light” and one “heavy” chain. The variable regions of each light/heavy chain pair form an antibody binding site. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains (CH). Each light chain has a variable domain at one end (VL) and a constant domain (CL) at its other end, wherein the constant domain of the light chain is aligned with the first constant domain of the heavy chain, and the light chain variable domain is aligned with the variable domain of the heavy chain. Light chains are classified as either lambda chains or kappa chains based on the amino acid sequence of the light chain constant region.

Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1m(f, z, a or x), G2m(n), G3m(g, b, or c), Am, Em, and Km(1, 2 or 3)). Immunoglobulins include, but are not limited to, monoclonal antibodies (mAb) (including full-length monoclonal antibodies), polyclonal antibodies, multi specific antibodies formed from at least two different epitope binding fragments (e.g., bispecific antibodies), CDR-grafted, human antibodies, humanized antibodies, camelised antibodies, chimeric antibodies, anti-idiotypic (anti-Id) antibodies, intrabodies, and desirable antigen binding fragments thereof, including recombinantly produced antibody fragments. Examples of antibody fragments that can be recombinantly produced include, but are not limited to, antibody fragments that include variable heavy- and light-chain domains, such as single-chain Fvs (scFv), single-chain antibodies, Fab fragments, Fab′ fragments, F(ab′)2 fragments. Antibody fragments can also include epitope-binding fragments or derivatives of any of the antibodies enumerated above. In suitable embodiments, the antibody product is a monoclonal antibody (mAb) and more suitably is a therapeutic antibody product.

As described herein, the antibody production process is suitably carried out in a bioreactor—a vessel suitable for the cultivation of producer cells that express the antibody of interest. Since the bioreactor is typically being used at a production scale, or a pilot scale prior to being scaled up for production, the bioreactor used in the production process typically has a volume of at least 10 L although smaller bioreactors may be used to test the process e.g. the AMBR® 250 system which has a volume of 100 to 250 mL. Accordingly, in exemplary embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Suitable reactors can be multi-use, single-use, disposable, or non-disposable and can be formed of any suitable material including metal alloys such as stainless steel (e.g., 316L or any other suitable stainless steel) and Inconel, plastics, and/or glass.

An antibody production process includes a cell culture or cell population which is producing the antibody product, along with a production medium or buffer, that suitably includes the necessary reagents and supplements, including suitable nutrient media, to support the cell proliferation and production of the desired antibody.

The total filled volume of a bioreactor, also referred to herein as the “production process volume,” refers to the actual volume of cell culture in the bioreactor during the production process. This is less than the total bioreactor volume but is also typically in the order of about 10 L to about 50000 L. Thus the exemplary volumes described above for the bioreactor volume are also applicable to the fill production process volume.

As described herein, the methods of modulating afucosulation include adding an amount of mannose to the bioreactor during the antibody production process. As used herein “mannose” refers to the sugar monomer of the aldohexose series of carbohydrates, and is a C-2 epimer of glucose. Mannose exists in both D and L isomeric forms but typically it is the D isomer that is used in the methods of the present invention:

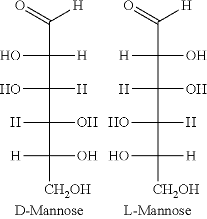

The methods described herein suitably include adding mannose to the bioreactor in which the antibody production process is taking place, and thus the mannose is itself added to the antibody production process. The amount of mannose (i.e. weight of mannose) that is added is calculated relative to the fill volume of the bioreactor, and thus the volume of the production process. Accordingly, the mannose-containing solution is added to bring the concentration in the cell culture fluid to the desired value. For example, 1 L of a 10 g/L mannose feedstock may be added to a bioreactor containing 9 L of cell culture fluid (media and cell biomass) to achieve a 1 g/L final concentration. The addition of the mannose can be carried out using any suitable process, including the addition via one or more valves or ports on a bioreactor, the addition of mannose directly to a bioreactor via an opening or top of the bioreactor, or the mannose can be added to a solution that is to be added to the bioreactor. For example, the mannose can be included in a nutrient media solution that is going to be introduced during the antibody production process, thereby also adding the mannose to the process. It can be added as a standalone solution or as a component of a multicomponent media feed. It should be noted that the methods described herein require the addition of mannose to the antibody production process, for the purpose of increasing afucosylation/controlling the level of afucosylation, rather than simply including mannose at a low amount in nutrient media to promote cell proliferation, etc.

The amount of mannose that is added will depend on the degree of afucosylation that is required as well as the ability of the cells to tolerate the addition of the mannose. Typically the amount of mannose to be added is at least 1 g/L, such as greater than about 2 g/L, greater than about 3 g/L, greater than about 4 g/L, greater than about 5 g/L, greater than about 6 g/L, greater than about 7 g/L, greater than about 8 g/L, greater than about 9 g/L. The amount of mannose added is typically less than about 20 g/L, such as less than about 15, 14, 13, 12, 11 or 10 g/L, or less than about 9 or 8 g/L or less than about 7 g/L. Adding too much mannose can lead to hyperosmolality which in turn can decrease cell growth. The amount of mannose added can therefore be from about 1 g/L to about 20 g/L, about 1 g/L to about 15 g/L, 14 g/L, 13 g/L, 12 g/L, 11 g/L, 10 g/L, 9 g/L, 8 g/L or 7 g/L; about 2 g/L to about 15 g/L, 14 g/L, 13 g/L, 12 g/L, 11 g/L, 10 g/L, 9 g/L, 8 g/L or 7 g/L; about 3 g/L to about 15 g/L, 14 g/L, 13 g/L, 12 g/L, 11 g/L, 10 g/L, 9 g/L, 8 g/L or 7 g/L. Larger amounts or smaller amounts of mannose can also be added to achieve the desired levels of afucosylation taking into account cell production performance. 1 g/L mannose is equivalent to 5.6 mM mannose.

In one embodiment, where maximum fucosylation is desired, the amount of mannose added will typically be towards the higher levels outlined above. In another embodiment where the goal is to match afucosylation to a target level for the antibody with reference to previous production processes, the addition of mannose may be at levels anywhere in the ranges stated above and may also be varied during the production process to achieved the desired matching levels.

The timing of the addition of mannose to the antibody production process can be varied based on the type of antibody being produced, the type of process being carried out, the bioreactor, the requirements of the production plant in which the process is being carried out, etc. Antibody production processes typically have a growth phase to achieve rapidly a desired cell density/biomass and then a production phase to encourage high specific productivity of the antibody of interest. In some processes the transition from growth phase to production phase is accompanied by a temperature shift.

To achieve the modulation of afucosylation of the final product, the mannose levels will be elevated during all or part of the production phase. This can be achieved by feeding mannose-containing media at various stages during the cell culture process. Typically, mannose will be added during all of the production phase but mannose can also be fed during the growth phase and then allowing the cells to produce the antibody of interest under elevated mannose levels during the production phase without adding further mannose.

The production is typically one of the two main production process methods known in the art: fed batch and perfusion processes, or a hybrid of the two.

In some embodiments, mannose is added within (i.e., during) the first 12-48 hours of the cell culture process, and more suitably within the first 12-36 hours, including within the first 12 hours, the first 24 hours, or the first 36 hours. In other embodiments, mannose is added towards the end of the growth phase, such as 12-24 hours prior to the production phase. The addition of the mannose can also take place multiple times throughout the cell culture process (growth phase and/or production phase (i.e., 2, 3, 4, 5, 6, 7, 8, 9, 10 times, etc.), with each addition being at a desired amount (i.e., g/L amount) to achieve a final desired amount of mannose added.

In one embodiment the mannose is added in fixed amounts according to a pre-defined strategy, like a recipe. For example the mannose can be included with one of the standard feeds and adopt the same timing regimen, or as a separate feed.

In another embodiment, the amount of mannose to be added is adjusted as a result of periodic measurements of the expressed antibody, e.g. if those measurements indicate that afucosylation is drifting from a pre-determined target value. The amount of mannose added would then be increased if afucosylation is decreasing and decreased if afucosylation is increasing relative to the target value. Measurements of afucosylation may be performed off line or in line.

A predetermined value may be based on the level of afucosylation obtained when the process was run on a previous occasion e.g. in a different size vessel, at a different site etc. It may also be based on an analysis of a commercially available product e.g. when producing a biosimilar of that product so that CQAs can be adhered to as closely as possible for regulatory purposes. A predetermined level may for example be expressed as a range (such as 4.0 to 4.5%) or as a midpoint with a tolerance (4.25%+/0.25%).

As described herein, where the purpose of the process is to increase afucosylation as a result of the addition of mannose to the antibody production process, the afucosylation of the antibody product that is produced by the antibody production process is typically increased by at least 0.5%, as compared to the same antibody product if produced by the antibody production process without the addition of mannose. That is, if the antibody product from the methods of production described herein (that include the addition of mannose) is compared to the same antibody product produced in the same antibody production process, but without the inclusion of the additional mannose, the antibody produced in the methods described herein exhibit afucosylation that is increased by at least 0.5%.

Measurement of the amount of increase of afucosylation of an antibody product prepared according to the present methods that include the addition of mannose, in comparison to the same antibody product produced with the addition of mannose, are made using methods known in the art, such as mass spectrometry analysis, including liquid chromatography-mass spectrometry, as well as other methods. As also described above, since there can be some variation between the methods used, in one embodiment the percentage is measured using a TOF LC/MS system: the antibodies are reduced and then loaded directly onto a liquid chromatograph mass spectrometer (LC-MS), such as an Agilent 6230B TOF LC/MS system (without the need for a PNGase F digestion), with the reduced antibodies being passed through a reverse phase de-salting column on the liquid chromatograph prior to being injected into the time of flight mass spectrometer.

Other suitable methods include those described in, for example, Tay and Butler, 2015, J. Biol. Methods 2:19 Mishra et al., 2020, J. Biotechnology X 5: 100015). In one of these described methods, glycans can be removed using PNGase F and dried. They are then labelled using 2-AB labelling and analysed using hydrophilic interaction liquid chromatography-HPLC (HILIC-HPLC).

Comparison of the amount of afucosylation (or fucosylation) from one protein population to another provides the percentage increase in afucosylation (or decrease in fucosylation) and is generally provided relative a population of antibodies. That is, the measurement of a percent increase in afucosylation from one antibody population compared to another is generally calculated based on an amount of antibody produced that is on the order of about 0.5 g/L or more, rather than on an individual antibody basis. Suitably, the amount of antibody produced using the methods described herein is about 1 g/L or more, suitably 5 g/L or more, or about 10 g/L or more. Thus, in embodiments herein where there is an increase in afucosylation of at least 0.5%, the increase is measured relative a total amount of antibody that is about 0.5 g/L or more.

In exemplary embodiments, the amount of increase of afucosylation resulting from the methods described herein is at least a 0.5% increase, or in other embodiments, at least a 0.6% increase, at least a 0.7% increase, at least a 0.8% increase, at least a 0.9% increase, at least a 1% increase, at least a 1.1% increase, at least a 1.2% increase, at least a 1.3% increase, at least a 1.4% increase, at least a 1.5% increase, at least a 1.6% increase, at least a 1.7% increase, at least a 1.8% increase, at least a 1.9% increase, at least a 2.0% increase, at least a 2.1% increase, at least a 2.2% increase, at least a 2.3% increase, at least a 2.4% increase, at least a 2.5% increase, at least a 2.6% increase, at least a 2.7% increase, at least a 2.8% increase, at least a 2.9% increase, at least a 3.0% increase, or an increase of 0.5% to about 2.0%, an increase of about 0.5% to about 1.5%, or an increase of about 0.5% to about 1.0%.

In another embodiment, the process is used to control the level of fucosylation to match a predetermined level (target value). Accordingly in one embodiment a method is provided for matching the afucosylation of a recombinantly produced antibody to a previously obtained target afucosylation percentage for the same antibody, the method comprising culturing cells that express the antibody in a bioreactor; controlling the addition of mannose to the bioreactor during the antibody production process to obtain the expressed antibody with the target percentage afucosylation.

Typically, the level of afucosylation is controlled to within +/−0.25% of the desired target value (where the target value is a range, the variation is with respect to the midpoint of the range). For example, the level of afucosylation is controlled to within +/−0.5%.

The antibody production processes used in the methods described herein are suitably carried out in mammalian cells, though in other embodiments, bacteria or insect cells can also be used to prepare the antibody products. Exemplary mammalian cells that can be used in the antibody production processes include human, mouse, rat, Chinese hamster, Syrian hamster, monkey, ape, dog, horse, ferret, and cat cells. In embodiments, the cells are Chinese hamster ovary (CHO) cells. In exemplary embodiments, the cells are CHO-K1 cells, CHOK1SV® cells, DG44 CHO cells, DUXB11 CHO cells, CHO-S, CHO GS knock-out cells (a CHO cell where all endogenous copies of the glutathione synthetase (GS) gene have been inactivated), CHOK1SV® FUT8 knock-out cells, CHOZN, or CHO-derived cells. An exemplary CHO GS knock-out cell (e.g., GS-KO cell) is a CHOK1SV® GS knockout cell (such as a GS Xceed® cell-CHOK1SV GS-KO®, Lonza Biologics, Inc.). A CHO FUT8 knockout cell is, for example, the Potelligent® CHOK1SV® FUT8 knock-out (Lonza Biologics, Inc.). In other embodiments, the cell may be from a cell line such as the mouse myeloma (NSO)-cell line, HT1080, H9, HEK293 cell line, HeLa cell line, a T-cell, or a HepG2, MCF7, MDBK Jurkat, NIH3T3, PC12, BHK (baby hamster kidney cell), VERO, SP2/0, YB2/0, Y0, C127, L cell, COS, etc.

In embodiments, the antibody production process is carried out in a fed-batch bioreactor, in which a fed-batch production process is used where a nutrient media is provided to the antibody production process and the product remains in the bioreactor until the end of the production run. In such embodiments, the addition of the mannose also takes place such that the antibody is not removed until the end of the production run.

In additional embodiments, the antibody production process is a perfusion process carried out in a perfusion bioreactor. Exemplary perfusion bioreactors for use in the methods described herein may comprise a fermenter, a stirred-tank reactor, or a wave-type bioreactor. In some cases, the perfusion bioreactor may comprise a hollow vessel or container that includes a bioreactor volume for receiving a cell culture within a fluid growth medium. In some instances, the perfusion bioreactor may be placed in association with a rotatable shaft coupled to an agitator for stirring the cell culture. The perfusion bioreactor can be made from various materials, such as stainless steel or other metal, a polymer (e.g., a rigid polymer or flexible polymer), or any combination thereof. In an embodiment, the perfusion bioreactor may include various components and equipment, such as baffles, spargers, gas supplies, heat exchangers, and the like which allow for the cultivation and propagation of cells within a cell culture. In embodiments in which a perfusion reaction (and perfusion bioreactor) are utilized, mannose may be added consistently throughout the process so as to maintain the desired level of added mannose.

In some embodiments, the perfusion process may be a steady state perfusion production process. In such cases, a perfusion bioreactor may continuously receive an input medium or media, such as a nutrient media, as well as mannose as desired, via at least the input port, while an outflow medium or media may be continuously removed from the perfusion bioreactor via at least the outflow port. The continuous introduction of one or more input components via an input medium or media and the continuous removal of effluent material or other material via the outflow medium or media may maintain a steady state or pseudo-steady-state condition within a cell culture contained within the bioreactor. For instance, the steady state condition may involve maintaining a relatively constant volume of cell culture and of a medium or media in which the cell culture is placed. As an example, the volume of the cell culture may be maintained such that it does not vary by more than, e.g., 10%, 8%, 5%, or 3%. In some implementations, the steady state perfusion production process may maintain a desired cell density within the perfusion bioreactor.

In embodiments, the perfusion bioreactor may include one or more ports, such as an input port and an outflow port. The input port may be configured to allow one or more input components to be fed into the perfusion bioreactor. In some cases, the one or more input components (also referred to as one or more input materials) may include one or more nutrients, such as glucose, vitamins, lipids, mannose, etc. In some cases, the one or more input components may be delivered into the perfusion bioreactor via a liquid or other medium that flows into the perfusion bioreactor, in which case the medium or media may be referred to as an input medium or input media. For instance, the input medium or input media may be a nutrient medium or media that is used by the cells to grow or expand within the perfusion bioreactor. In an embodiment, the outflow port may be configured to allow material to be removed from the perfusion bioreactor. For instance, the material that is removed may include a waste, by-product, or other spent material (also referred to as an effluent material). In some cases, such a material may be removed via a liquid or other medium or media that flows out of the perfusion bioreactor. Such a medium or media may be referred to as an outflow medium or media. In some implementations, a desired bioproduct may be harvested from the bioreactor via the same outflow port, or the bioreactor may have another port for harvesting of the bioproduct.

In further embodiments, provided herein is a method for increasing afucosylation of an mAb antibody product. The method suitably includes: providing an mAb antibody production process carried out in a mammalian cell in a bioreactor, the production process having a volume of at least 50 L; adding an amount of mannose to the bioreactor greater than about 5 g/L during the mAb antibody production process; and producing the mAb antibody product via the antibody production process, wherein afucosylation of the mAb antibody product is increased by at least 2%, as compared to the mAb antibody product produced by the mAb antibody production process without the addition of mannose.

In exemplary embodiments, the mAb antibody production process is carried out in a fed-batch bioreactor. In other embodiments, the antibody production process is carried out in a perfusion bioreactor. Suitably, the antibody production process is a perfusion process.

Once biosynthesis of the product by the production cells has progressed to a satisfactory point, the product can be harvested e.g. withdrawing culture medium and separating the supernatant from cells and cell debris. The product can be subjected to one or more purification/treatment steps to obtain purified product, such as affinity chromatography, ion exchange chromatography, filtration and/or viral inactivation. The product may also be combined with one or more pharmaceutically acceptable carriers, excipients or diluents to produce a composition such as a formulated pharmaceutical composition e.g. with one or more of a buffer, a surfactant, a stabilizer (such as trehalose, sucrose, glycerol), an amino acid (such as glycine, histidine, arginine), metal ions/chelators, salts and/or a preservative.

### EXAMPLES

### Modulation of Afucosylation With the Addition of Mannose

CHO GS-KO cells were cultured in chemically defined culture medium supplemented with different chemicals at different concentrations to test the impact of those chemicals on the N-linked glycan profile of the product produced by the cultures. The product produced by this cell line was a model IgG antibody.

### Experiment Flow:

1. Prepare culture media with supplemented chemicals
2. Culture cells in media from (1)
3. Harvest mAb produced in cultures and measure glycan profile of purified, reduced, and de-salted mAb products
4. Determine statistical impact of chemical supplements on glycan product quality

### Preparation of Media With Supplemented Chemicals:

The chemicals that were supplemented to the medium included:
None (Control)
Mannose
N-Acetylmannosamine (ManNAc)+Galactose

Concentrated stock solutions of mannose, ManNAc, and galactose were prepared and added into culture media. Specific volumes of the stock solution was supplemented to the culture media to generate the conditions in Table 1:

TABLE 1

| Condition | [Supplement] |
|---|---|
| culture medium (Control) | None |
| culture medium + Mannose | 1, 3, 6 g/L Mannose |
| culture medium + 1 mM Galactose + | 0.1, 1.0, 10 mM ManNAc |
| ManNAc | |

### Cell Culture:

GS-KO cells were inoculated at a density of 2×10⁵ cells/mL into vented shake flasks containing medium from Table 1. Cultures were grown for five days in a temperature, CO₂, and humidity-controlled incubator. Daily samples were taken to monitor the culture health.

### Product Harvest & Glycan Measurement:

mAb product was harvested from day 5 shake flask cultures and purified through Protein A capture. Purified mAb was prepared for glycan analysis by reducing mAb to separate heavy and light chains. Reduced mAb was injected onto an LC-MS where the reduced mAb was de-salted by passing through a reverse-phase de-salting column on the LC prior to injection into a time of flight mass spectrometer (TOF MS) (Agilent 6230B). Three injections per sample was performed for technical replicates.

### Glycan Data Analysis and Statistical Analysis:

Protein Metrics Software was used to process the resulting LC-MS data and the relative abundance of each glycan species was reported. The glycan species measured in processing included: G0, G0F, G1F, G2F, G1F+NeuAc, G2F+NeuAc, G2F+2NeuAc. Percent afucosulated mAb was calculated by dividing the total amount of afucosylated species by the sum of afucosylate and fucosylated species (G0/G0F+G0). GraphPad Prism, Dunnett post-hoc analysis was performed to determine if mAb produced in cultures fed mannose-supplemented media or galactose/ManNAc- supplemented media significantly differed from mAb produced in cultures fed unsupplemented medium (control).

### Results/Discussion

Supplementation of culture medium with mannose but not galactose and ManNAc significantly increased the % afucosylated mAb. Increasing concentrations of mannose caused a concomitant increase in afycosylated species in a linear relationship. While supplementation of higher concentrations of ManNAc (10 uM) did produce a statistically different result compared to the control, with ManNAc decreasing afucosylation slightly.

TABLE 2

| | | Std. Dev. | |
|---|---|---|---|
| | | (n = 3 | |
| | Average % | technical | Significance level |
| Condition | Afucosylation | replicates) | compared to Control |
| culture medium (Control) | 5.21% | 0.10% | |
| culture medium + 1 g/L | 5.63% | 0.12% | * p value = 0.01-0.05 |
| Mannose | | | |
| culture medium + 3 g/L | 6.10% | 0.20% | *** p value < 0.001 |
| Mannose | | | |
| culture medium + 6 g/L | 7.27% | 0.10% | *** p value < 0.001 |
| Mannose | | | |
| culture medium + 1 mM | 5.22% | 0.21% | ns |
| Galactose + 0.1 uM ManNAc | | | |
| culture medium + 1 mM | 4.66% | 0.47% | ns |
| Galactose + 1.0 uM ManNAc | | | |
| culture medium + 1 mM | 4.59% | 0.04% | * p value = 0.01-0.05 |
| Galactose + 10 uM ManNAc | | | |

### EXEMPLARY EMBODIMENTS

Embodiment 1 is method for increasing afucosylation of an antibody recombinantly expressed in a bioreactor, the method comprising: culturing cells that express the antibody in a bioreactor; adding mannose to cell culture during the antibody production process such that the afucosylation of the antibody is increased as compared to the same antibody produced by the antibody production process without the addition of mannose.

Embodiment 2 includes the method of embodiment 1, wherein the amount of mannose added is from about 1 g/L to about 10 g/L.

Embodiment 3 includes the method of embodiment 1 or embodiment 2, wherein the bioreactor has a volume of at least 10 L.

Embodiment 4 includes the method of any of embodiments 1-3, wherein the antibody is a monoclonal IgG.

Embodiment 5 includes the method of any of embodiments 1-4, wherein the cells are mammalian.

Embodiment 6 includes the method of any of embodiments 1-5, wherein afucosylation of the antibody, is increased by at least 0.5% as compared to the antibody produced by the antibody production process without the addition of mannose.

Embodiment 7 includes the method of embodiment 6, wherein afucosylation of the antibody product is increased by at least 1%, such as at least 2%, as compared to the antibody produced by the antibody production process without the addition of mannose.

Embodiment 8 includes the method of any of embodiments 1-7, wherein the antibody production process is a fed-batch process.

Embodiment 9 includes the method of any of embodiments 1-7, wherein the antibody production process is a perfusion process.

Embodiment 10 includes the method of any of embodiments 1-9 which further comprises isolating the expressed antibody and optionally subjecting the antibody to one or more purification steps.

Embodiment 11 is a method for matching the afucosylation of a recombinantly produced antibody to a previously obtained target afucosylation percentage for the same antibody, the method comprising: culturing cells that express the antibody in a bioreactor; controlling the addition of mannose to the bioreactor during the antibody production process to obtain the expressed antibody with the target percentage afucosylation.

Embodiment 12 includes the method of embodiment 11 wherein the percentage afucosylation of the expressed antibody is within +/−0.25% of the target percentage afucosylation.

Embodiment 13 includes the method of embodiment 11 or embodiment 12, wherein the antibody production process is a perfusion process.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. A method for increasing afucosylation of an antibody recombinantly expressed in a bioreactor, the method comprising:
culturing cells that express the antibody in a bioreactor;
adding mannose to cell culture during the antibody production process such that the afucosylation of the antibody is increased as compared to the same antibody produced by the antibody production process without the addition of mannose.

2. The method of claim 1, wherein the amount of mannose added is from about 1 g/L to about 10 g/L.

3. The method of claim 1, wherein the bioreactor has a volume of at least 10 L.

4. The method of claim 1, wherein the antibody is a monoclonal IgG.

5. The method of claim 1, wherein the cells are mammalian.

6. The method of claim 1, wherein afucosylation of the antibody, is increased by at least 0.5% as compared to the antibody produced by the antibody production process without the addition of mannose.

7. The method of claim 6, wherein afucosylation of the antibody product is increased by at least 1%, such as at least 2%, as compared to the antibody produced by the antibody production process without the addition of mannose.

8. The method of claim 1, wherein the antibody production process is a fed-batch process.

9. The method of claim 1, wherein the antibody production process is a perfusion process.

10. The method of claim 1, which further comprises isolating the expressed antibody and optionally subjecting the antibody to one or more purification steps.

11. A method for matching the afucosylation of a recombinantly produced antibody to a previously obtained target afucosylation percentage for the same antibody, the method comprising:
culturing cells that express the antibody in a bioreactor;
controlling the addition of mannose to the bioreactor during the antibody production process to obtain the expressed antibody with the target percentage afucosylation.

12. The method according to claim 11 wherein the percentage afucosylation of the expressed antibody is within +/−0.25% of the target percentage afucosylation.

13. The method of claim 11, wherein the antibody production process is a perfusion process.
